(19) United States
(12) Patent Application Publication
(10) Pub. No.: US 2021/0325356 A1
(43) Pub. Date: Oct. 21, 2021

(54) METHOD OF ESTABLISHING FINGERPRINT SPECTRUM OF TRADITIONAL CHINESE MEDICINE COMPOSITION WITH EFFECT OF REGULATING DEPRESSION EMOTION OR FORMULATION THEREOF

(71) Applicants: CHENLAND NUTRITIONALS, INC., Pomona, CA (US); QINGDAO CHENLAND PHANMACEUTICAL TECHNOLOGY DEVELOPMENT CO., LTD, Qingdao, Shandong (CN)

(72) Inventors: Shengcan ZOU, Qingdao, Shandong (CN); Zengliang ZHANG, Qingdao, Shandong (CN); Wenyu LI, Qingdao, Shandong (CN); Shanglong WANG, Qingdao, Shandong (CN); Lei ZONG, Qingdao, Shandong (CN); Jiancheng ZONG, Qingdao, Shandong (CN)

(73) Assignee: CHENLAND NUTRITIONALS, INC., Pomona, CA (US)

(21) Appl. No.: 17/043,732

(22) PCT Filed: Dec. 20, 2019
(86) PCT No.: PCT/CN2019/126932

§ 371 Date: Sep. 30, 2020

(30) Foreign Application Priority Data
Nov. 14, 2019 (CN) 201911111679.0

(51) Int. Cl. G01N 30/86; G01N 30/30; G01N 30/32; G01N 33/15; G01N 30/74; G01N 30/06

(52) U.S. Cl. CPC G01N 30/8686; G01N 30/8631; G01N 30/30; G01N 2030/027; G01N 33/15; G01N 30/74; G01N 30/06; G01N 30/32

(57) ABSTRACT

The present invention relates to the field of analysis of traditional Chinese medicine, and in particular to a method of establishing a fingerprint spectrum of a traditional Chinese medicine composition with effect of regulating depression emotion or a formulation thereof, wherein the traditional Chinese medicine composition consists of the following traditional Chinese medicine extracts in parts by weight: 40˜80 parts of Hypericum perforatum extract, 10˜40 parts of Acanthopanax senticosus extract, and 2˜20 parts of tree peony root bark extract; and the method comprises establishing the fingerprint spectrum by high performance liquid chromatography, and the chromatographic conditions are as follows: an octadecylsilane bonded silica gel chromatographic column is used as a chromatographic column; column temperature is 25˜35° C.; flow rate is 0.5˜1.1 mL/min; detection wavelength is 290˜310 nm; mobile phase A is phosphoric acid aqueous solution, mobile phase B is acetonitrile, and a gradient elution program is used.

This application claims the priority of the Chinese Patent Application No. 201911111679.0 filed with the Chinese Patent Office on Nov. 14, 2019, titled “method of establishing fingerprint spectrum of traditional Chinese medicine composition with effect of regulating depression emotion or formulation thereof”, the entire contents of which are incorporated herein by reference.

### FIELD

The present invention relates to the field of analysis of traditional Chinese medicine, and in particular to an method of establishing a fingerprint spectrum of a traditional Chinese medicine composition with the effect of regulating depression emotion or a formulation thereof.

### BACKGROUND

Depression emotion is a common mood disorder disease, which seriously endangers people's physical and mental health. With the development of society, the pace of life has accelerated, competition for employment has intensified, and the number of people experiencing depression emotion has increased year by year. Severe depression emotion will develop into depression, people suffered from which are very suicidal, with a suicide rate of about 15%. In addition, about more than 90% of patients with mood disorders clinically can have sleep disorders in a certain period of time. Therefore, effective prevention and treatment of sleep disorders is of great significance for the relief and improvement of depression emotion. Currently, among the drugs used to treat depression, chemical drugs have obvious curative effects, but they have shortcomings such as narrow antidepressant spectrum, large adverse reactions, and easy recurrence. Therefore, people are turning to traditional Chinese medicine to fight depression.

Hypericum perforatum is bitter, astringent, and neutral in nature, and the action thereof is localized to the liver meridian. When used as a medicine, Hypericum perforatum has the effect of astringency and hemostasis, regulating menstruation and promoting lactation, clearing away heat and toxic materials, and removing dampness; and it mainly aims at hemoptysis, hematemesis, intestinal wind bleeding, metrorrhagia and metrostaxis, traumatic bleeding, irregular menstruation, breast milk stoppage for lactating women, jaundice, sore throat, swelling and pain of eye, urinary tract infection, mouth and nose sores, carbuncle furuncle swelling poison, and burn and scald. In the early 19th century, researchers discovered that Hypericum perforatum can be used to treat mood disorders. In the past two centuries, researches on the treatment of mental diseases of Hypericum perforatum are still ongoing.

Acanthopanax senticosus is pungent, slightly bitter, and warm in nature, and the action thereof is localized to the spleen, kidney and heart meridian. It has the effect of benefiting qi to invigorate the spleen and invigorating kidney for tranquillization and can be used for treating yang deficiency of spleen and kidney, physical weakness, loss of appetite, waist and knee pain, and insomnia and dreaminess. Acanthopanax senticosus has a long history of being widely used as a medicine in traditional Chinese medicine and has the function of “invigorating spleen-stomach and replenishing essence, strengthening the muscles and bones, and strengthening the mental power”. Acanthopanax senticosus can “lighten body and endure aging” after taking it for a long time, and the combination with other medicines can also “promote the diet, invigorate the strength, and not forgetful”. In addition, clinical studies have confirmed that Acanthopanax senticosus, like the traditional antidepressants imipramine, fluoxetine, etc., can help improve the mood and emotion of patients with depression, with fewer side effects and is safer and more effective.

Tree peony root bark is bitter, pungentin and slightly cold, and has the effects of clearing heat and cooling blood, promoting blood circulation to remove blood stasis. Studies have found that intraperitoneal injection or oral administration of paeonol in mice has sedative, hypnotic, and analgesic effects. Furthermore, paeonol can reduces the body temperature of normal mice (intraperitoneal injection or gavage) and also has an antipyretic effect on mice with artificial fever (caused by the injection of Bacillus typhi and Bacillus paratyphosus); and it is also resistant to shock induced by electric shock or drug.

In the present invention, the Hypericum perforatum extract, the Acanthopanax senticosus extract and the tree peony root bark extract are combined according to specific dosages, and the composition has a remarkable effect on treating depression.

The fingerprint spectrum of the traditional Chinese medicine has the characteristics of overall, macroscopic, and fuzzy analysis. It can be used to describe the overall characteristics of the traditional Chinese medicine and employ appropriate fuzzy processing modes to achieve the purpose of overall quality control. Therefore, it has become an effective means of quality control of the traditional Chinese medicine. Among them, chromatographic fingerprint spectrum analysis can visualize the overall characteristics of the various chemical components contained in traditional Chinese medicines, thereby revealing quality problems that are difficult to find in routine tests. Fingerprint spectrum is a modern quality control method of traditional Chinese medicine from the perspective of “full ingredients” based on the characteristics of the overall comprehensive action of multi-component and multi-targets of traditional Chinese medicine, which provides more comprehensive quality control for non-single component medicines. At present, there is a lack of qualitative research on this traditional Chinese medicine formula composition system for improving depression emotion and sleep. Therefore, it is necessary to establish the fingerprint spectrum of this traditional Chinese medicine formula composition, and complete the qualitative analysis of this traditional Chinese medicine formula composition, so as to effectively control the quality of this Chinese medicine formula composition.

### SUMMARY

In view of this, the present invention provides a method of establishing a fingerprint spectrum of a traditional Chinese medicine composition with the effect of regulating depression emotion or a formulation thereof. The fingerprint spectrum obtained by the method of establishing the fingerprint spectrum comprehensively reflects the quality information of the traditional Chinese medicine composition, thereby achieving the purpose of more comprehensively and effectively controlling the product quality of this traditional Chinese medicine composition.

In order to achieve the above objects of the invention, the present invention provides the following technical solutions:

The present invention provides a method of establishing a fingerprint spectrum of a traditional Chinese medicine composition with the effect of regulating depression emotion or a formulation thereof, wherein the traditional Chinese medicine composition consists of the following traditional Chinese medicine extracts in parts by weight: 40˜80 parts of Hypericum perforatum extract, 10˜40 parts of Acanthopanax senticosus extract, and 2˜20 parts of tree peony root bark extract.

The fingerprint spectrum is constructed by high performance liquid chromatography for a tested solution and a reference solution respectively and the chromatographic conditions are as follows: an octadecylsilane bonded silica gel chromatographic column is used as a chromatographic column; column temperature is 25˜35° C.; flow rate is 0.5˜1.1 mL/min; detection wavelength is 290˜310 nm; and mobile phase A is 0.05%˜0.15% phosphoric acid aqueous solution, mobile phase B is acetonitrile, and gradient elution program is as follows:

| Time/min | A/% | B/% |
|---|---|---|
| 0 | 90 | 10 |
| 8 | 86 | 14 |
| 12 | 86 | 14 |
| 20 | 80 | 20 |
| 30 | 75 | 25 |
| 40 | 60 | 40 |
| 42 | 40 | 60 |
| 50 | 40 | 60 |
| 52 | 90 | 10 |
| 60 | 90 | 10 |

The object of the present invention is to overcome the shortcomings in the prior art, that is, the traditional Chinese medicine compound preparations basically have only one or two index ingredients in the content control, the quality control way is single, the index is limited, and it is difficult to comprehensively reflect the quality status of the variety of the multiple materials, multiple components, and multiple targets, thereby the provision of the method of establishing a fingerprint spectrum of the traditional Chinese medicine composition with the effect of regulating depression emotion and the fingerprint spectrum can provide a reference for the overall control and evaluation of the quality of the traditional Chinese medicine formula composition for improving depression emotion and sleep.

Preferably, the detection wavelength is 300 nm.

Preferably, the mobile phase A is 0.1% phosphoric acid aqueous solution.

Preferably, the chromatographic column is Agilent TC-C18 chromatographic column of 250×4.6 mm, 5 μm.

Preferably, the column temperature is 30° C.

Preferably, the flow rate is 1.0 mL/min.

Preferably, the method of preparing the tested solution comprises steps of: mixing the traditional Chinese medicine composition or the formulation thereof with 40 vt %˜60 vt % methanol aqueous solution at a ratio of the traditional Chinese medicine composition or the formulation thereof to the methanol aqueous solution in g/mL of (0.5˜2.0): 25, performing ultrasonic treatment and filtration, and collecting subsequent filtrate.

Preferably, the method of preparing the reference solution comprises steps of: mixing hyperoside reference substance or paeonol reference substance with 40 vt %˜60 vt % methanol aqueous solution at a ratio of the hyperoside reference substance or the paeonol reference substance to the methanol aqueous solution in g/mL of (0.5˜1.5):1.

Preferably, the traditional Chinese medicine composition consists of the following traditional Chinese medicine extracts in parts by weight: 65˜80 parts of Hypericum perforatum extract, 10˜35 parts of Acanthopanax senticosus extract, and 2˜10 parts of tree peony root bark extract.

Preferably, the traditional Chinese medicine composition consists of the following traditional Chinese medicine extracts in parts by weight: 64˜77 parts of Hypericum perforatum extract, 13.3˜25 parts of Acanthopanax senticosus extract, and 2.7˜10 parts of tree peony root bark extract.

More preferably, the traditional Chinese medicine composition consists of the following traditional Chinese medicine extracts in parts by weight: 77 parts of Hypericum perforatum extract, 20 parts of Acanthopanax senticosus extract, and 3 parts of tree peony root bark extract.

Preferably, the method of preparing the traditional Chinese medicine composition comprises the following steps:

(1) to Hypericum perforatum, adding 10˜12 times weight of 60 vt %˜80 vt % ethanol aqueous solution containing 0.05%˜2% sodium hydroxide, extracting under heating and refluxing 2 times for 1.5˜2.0 hours each time, combining the two filtrates and filtering, concentrating the filtrate to an extract with a relative density of 1.11˜1.13 at 72° C., and performing spray-drying to obtain Hypericum perforatum extract;

(2) to Acanthopanax senticosus pieces, adding 8˜10 times weight of 0 vt %˜80 vt % ethanol aqueous solution, extracting under heating and refluxing 3 times for 1.0˜2.5 hours each time, combining the three filtrates and filtering, concentrating the filtrate to an extract with a relative density of 1.11˜1.13 at 72° C., and performing spray-drying to obtain Acanthopanax senticosus extract;

(3) to tree peony root bark, adding 10˜14 times weight of water, heating and recovering 8˜10 times amount of distillate, refrigerating for 20˜24 hours, crystallizing, filtering, and performing low temperature drying to obtain tree peony root bark extract; and

(4) mixing the Hypericum perforatum extract, the Acanthopanax senticosus extract and the tree peony root bark extract according to a ratio.

Preferably, the method of preparing the traditional Chinese medicine composition comprises the following steps:

(1) to Hypericum perforatum, adding 10 times weight of 80 vt % ethanol aqueous solution containing 0.1% sodium hydroxide, extracting under heating and refluxing 2 times for 1.5 hours each time, combining the two filtrates and filtering, concentrating the filtrate to an extract with a relative density of 1.12 at 72° C., and performing spray-drying to obtain Hypericum perforatum extract;

(2) to Acanthopanax senticosus pieces, adding 10 times weight of the aqueous solution, extracting under heating and refluxing 3 times for 1.0 hours each time, combining the three filtrates and filtering, concentrating the filtrate to an extract with a relative density of 1.12 at 72° C., and performing spray-drying to obtain Acanthopanax senticosus extract;

(3) to tree peony root bark, adding 14 times weight of water, heating and recovering 9 times its amount of distillate, refrigerating for 24 hours, crystallizing, filtering, and performing low temperature drying to obtain tree peony root bark extract; and

(4) mixing the Hypericum perforatum extract, the Acanthopanax senticosus extract and the tree peony root bark extract according to a ratio.

The invention provides a method of establishing a fingerprint spectrum of a traditional Chinese medicine composition with the effect of regulating depression emotion or a formulation thereof. For the establishment method, the traditional Chinese medicine composition consists of the following traditional Chinese medicine extracts in parts by weight: 40˜80 parts of Hypericum perforatum extract, 10˜40 parts of Acanthopanax senticosus extract, and 2˜20 parts of tree peony root bark extract; and the fingerprint spectrums are established respectively by high performance liquid chromatography for a tested solution and an reference solution. and the chromatographic conditions are as follows: an octadecylsilane bonded silica gel chromatographic column is used as a chromatographic column; column temperature is 25˜35° C.; flow rate is 0.5˜1.1 mL/min; detection wavelength is 290˜310 nm; mobile phase A is 0.05%˜0.15% phosphoric acid aqueous solution, mobile phase B is acetonitrile, and gradient elution is used. The present invention has the following technical effects:

1. The fingerprint spectrum obtained by the method of establishing the fingerprint spectrum of the traditional Chinese medicine composition with the effect of regulating depression emotion provided by the present invention comprehensively reflects the quality information of the traditional Chinese medicine composition, thereby achieving the purpose of more comprehensively and effectively controlling the product quality of this traditional Chinese medicine composition.

2. In the method of establishing the fingerprint spectrum of the traditional Chinese medicine composition with the effect of regulating depression emotion provided by the present invention, the Similarity Evaluation System for Chromatographic Fingerprint of the Traditional Chinese Medicine provided by the Chinese Pharmacopoeia Commission is employed to identify the measured fingerprint, which is convenient and rapid to operate. Moreover, when the resulting similarity results are used to evaluate the fingerprint spectrum of the preparation, the conclusion is relatively objective and accurate.

3. In the method of establishing the fingerprint spectrum of the traditional Chinese medicine composition with the effect of regulating depression emotion provided by the present invention, the determination conditions of the fingerprint spectrum were established and the methodology was investigated by investigating the preparation method of the tested solution and systematically optimizing the conditions for determining the fingerprint spectrum such as the instrument, chromatographic column, mobile phase, and detection wavelength. Based on the test results of the fingerprint spectrums of multiple batches of the traditional Chinese medicine composition, the data were gradually accumulated and a standard fingerprint spectrum was proposed to be used as the fingerprint spectrum standard of the product, so as to achieve the purpose of being able to control the quality of the preparation more comprehensively and effectively.

4. In the method of establishing the fingerprint spectrum of the traditional Chinese medicine composition with the effect of regulating depression emotion provided by the present invention, the Similarity Evaluation System for Chromatographic Fingerprint of the Traditional Chinese Medicine provided by the Chinese Pharmacopoeia Commission is employed as the software for calculating the similarity of fingerprint spectrums of this traditional Chinese medicine compositions. The evaluation conclusions obtained by many experiments are basically the same, compared with the method of calculating the relative retention time and relative peak area. The Similarity Evaluation System for Chromatographic Fingerprint of the Traditional Chinese Medicine is used to evaluate the similarity of fingerprint spectrums, which is convenient and rapid to operate. Moreover, when the resulting similarity results are used to evaluate the fingerprint spectrum of the preparation, the conclusion is relatively objective and accurate.

5. The method of establishing the fingerprint spectrum of the traditional Chinese medicine composition with the effect of regulating depression emotion provided by the present invention employs the fingerprint spectrum to more comprehensively characterize the quality information of the composition, reflects the product quality as a whole, and employs multi-wavelength switching technology to establish content control indicators for all three medicinal materials. Furthermore, the preparation method of the tested solution is simple and convenient, and the test results are accurate and reliable. In addition, it was proposed to comprehensively control the quality of the traditional Chinese medicine composition by means of the fingerprint spectrum combined with multi-index component content control.

6. The method of establishing the fingerprint spectrum of the traditional Chinese medicine composition with the effect of regulating depression emotion provided by the present invention proposes to use the fingerprint spectrum to determine multiple index components in the traditional Chinese medicine composition in combination with multi-wavelength switching technology.

7. The method of establishing the fingerprint spectrum of the traditional Chinese medicine composition with the effect of regulating depression emotion provided by the present invention can provide a more comprehensive quality control method for this traditional Chinese medicine composition.

### BRIEF DESCRIPTION OF DRAWINGS

FIG. 1 shows the fingerprint spectrum of the traditional Chinese medicine composition of the present invention;

FIG. 2 shows the fingerprint spectrum of Hypericum perforatum;

FIG. 3 shows the fingerprint spectrum of Acanthopanax senticosus;

FIG. 4 shows the fingerprint spectrum of paeonol.

### DETAILED DESCRIPTION

The invention discloses a method of establishing a fingerprint spectrum of a traditional Chinese medicine composition with the effect of regulating depression emotion or a formulation thereof, which can be achieved by those skilled in the art through appropriately improving the process parameters in light of the present disclosure. In particular, it should be noted that, all similar substitutions and modifications will be apparent to those skilled in the art, and are all considered to be included in the present invention. Although the method and use of the present invention have been described by means of preferred examples, it is apparent to those skilled in the art that modifications or proper alterations and combinations can be made to the method and use described herein without departing from the content, spirit and scope of the present invention, so as to achieve and apply the technique of the present invention.

The invention discloses a method of establishing a fingerprint spectrum of a traditional Chinese medicine composition with the effect of regulating depression emotion and the fingerprint spectrum, belonging to the field of the analysis of traditional Chinese medicine.

The traditional Chinese medicine composition with the effect of regulating depression emotion is prepared by the following method:

to 77% of Hypericum perforatum, 20% of Acanthopanax senticosus, and 3% of Paeonol, 50% ethanol in parts by weight are added, extracted twice, filtered, and the two filtrates are combined and concentrated to an extract with a relative density of 1.11˜1.13 at 72° C., and then maltodextrin is added, mixed, dried, and granulated.

The tested solution is prepared as follows:

the traditional Chinese medicine composition is mixed well and porphyrized, and then 0.5˜2.0 parts by weight are placed in a triangular container with a plug, and 25 parts by volume of 50% methanol are added, weighed, ultrasonically processed, cooled down, and then weighed. 50% methanol is added to make up the lost weight, shook well, filtered, and the subsequent filtrate is collected.

The reference solution is prepared as follows:

an appropriate amount of hyperoside or paeonol reference substance is weighed, and 50% methanol is added to obtain a solution containing 1 part by weight per 1 part by volume.

The invention employs high performance liquid chromatography to establish fingerprint spectrum. In addition, an octadecylsilane bonded silica gel chromatographic column is used as a chromatographic column; column temperature: 25˜35° C.; flow rate: 0.5˜1.1 mL/min; detection wavelength: 300 nm; and 0.1% phosphoric acid solution A-acetonitrile B are used as the mobile phases, and gradient elution is performed in the following order:

| time/min | 0.1% phosphoric acid/% | acetonitrile/% |
|---|---|---|
| 0 | 90 | 10 |
| 8 | 86 | 14 |
| 12 | 86 | 14 |
| 20 | 80 | 20 |
| 30 | 75 | 25 |
| 40 | 60 | 40 |
| 42 | 40 | 60 |
| 50 | 40 | 60 |
| 52 | 90 | 10 |
| 60 | 90 | 10 |

The fingerprint spectrum of the present invention comprehensively reflects the quality information of the traditional Chinese medicine composition, thereby achieving the purpose of more comprehensively and effectively controlling the product quality of this traditional Chinese medicine composition.

The fingerprint spectrum comprises 11 common peaks, and the relative retention time of each peak and the reference peak are respectively: 8.291±0.05 min (Peak 1), 11.481±0.05 min (Peak 2), 12.173±0.05 min (Peak 3), 13.061±0.05 min (Peak 4), 25.763±0.05 min (Peak 5), 26.717±0.05 min (Peak 6), 27.303±0.05 min (Peak 7), 30.777±0.05 min (Peak 8), 31.521±0.05 min (Peak 9), 41.037±0.05 min (Peak 10), 45.426±0.05 min (Peak 11), of which Peak 6 is hyperoside.

The reagents or instruments used in the method of establishing a fingerprint spectrum of a traditional Chinese medicine composition with the effect of regulating depression emotion or a formulation thereof provided by the present invention are all commercially available.

The present invention will be further illustrated by the following examples:

### Example 1 Preparation of a Traditional Chinese Medicine Composition with the Effect of Regulating Depression Emotion

The traditional Chinese medicine formula composition for improving depression emotion and sleep involved in the present invention mainly consists of Hypericum perforatum extract, Acanthopanax senticosus extract and tree peony root bark extract. The preparation method of the composition comprises:

to Hypericum perforatum, 10 times of amount of 80% ethanol containing 0.1% sodium hydroxide was added, extracted 2 times under heating and refluxing for 1.5 hours each time. The two filtrates were combined, filtered, and concentrated to an extract with a relative density of about 1.12 (72° C.). The extract was spray-dried to obtain a dry extract powder;

after Acanthopanax senticosus medicinal material was broken into pieces, 10 times of amount of aqueous solution was added, extracted 3 times under heating and refluxing for 1.0 hour each time. Then the decoction was combined and filtered, and the filtrate was concentrated to an extract with a relative density of 1.12 (72° C.). The extract was spray-dried to obtain a dry extract powder; and

to tree peony root bark medicinal material, 14 times of amount of water was added, heated to recover 9 times of amount of distillate, refrigerated for 24 hours, crystallized, filtered, and low temperature dried. The above three extract powders were mixed according to a ratio of 77%:20%:3% of the three component Hypericum perforatum extract, Acanthopanax senticosus extract, and paeonol extract, to obtain the composition.

### Example 2 Preparation of Hypericum perforatum Extract

After Hypericum perforatum medicinal material was broken into pieces, 10 times of amount of 70% ethanol was added, extracted 2 times under heating and refluxing for 1.5 hours each time. The two filtrates were combined, filtered, and concentrated to an extract with a relative density of about 1.12 (72° C.). The extract was spray-dried to obtain Hypericum perforatum extract.

### Example 3 Preparation of Acanthopanax senticosus Extract

After Acanthopanax senticosus medicinal material was broken into pieces, 10 times of amount of 50% ethanol was added, extracted 3 times under heating and refluxing for 1.0 hour each time. Then the decoction was combined and filtered, and the filtrate was concentrated to an extract with a relative density of 1.12 (72° C.). The extract was spray-dried to obtain Acanthopanax senticosus extract.

### Example 4 Preparation of Tree Peony Root Bark Extract

to tree peony root bark medicinal material, 14 times of amount of water was added, heated to recover 9 times of amount of distillate, refrigerated for 24 hours, crystallized, filtered, and low temperature dried to obtain tree peony root bark extract.

### Example 5 Establishment of a Reference Fingerprint Spectrum of the Traditional Chinese Medicine Formula Composition for Improving Depression Emotion and Sleep

1. Sample: A reference substance of the traditional Chinese medicine formula composition for improving depression emotion and sleep was provided by Qingdao Silver Century Health Industry Group Co., Ltd.

2. Reagents: acetonitrile (HPLC, Wokai); methanol (HPLC, Wokai); phosphoric acid was analytically pure; and UPLC purified water was self-made by Milli-Q Pure Water System.

3. Instrument: Agilent Infinity 1260 (CA, USA).

4. Preparation of the reference solution of the composition: 0.71 g of the traditional Chinese medicine formula composition was placed into a 100 mL triangular conical flask, and 25 mL of 50% CH₃OH was added, accurately weighed, ultrasonicated for 20 min, and cooled to room temperature before weighing. 50% CH₃OH was added to make up the lost weight, shook well, filtered, and then an appropriate amount of filtrate was taken and passed through a 0.45 m microfiltration membrane to obtain the reference solution.

5. Chromatographic conditions: Chromatographic column was Agilent TC-C18 column (250×4.6 mm 5 μm), detection wavelength was 300 nm, column temperature was 30° C., flow rate was 1.0 mL/min, and mobile phases were acetonitrile-0.1% phosphoric acid aqueous solution with 0.1% phosphoric acid aqueous solution as A and acetonitrile as B. The injection volume was 10 μL, the analysis time was 60 min, and gradient elution was performed according to the following elution program:

TABLE 1
elution program

| time/min | A 0.1% phosphoric acid/% | B acetonitrile/% |
|---|---|---|
| 0 | 90 | 10 |
| 8 | 86 | 14 |
| 12 | 86 | 14 |
| 20 | 80 | 20 |
| 30 | 75 | 25 |
| 40 | 60 | 40 |
| 42 | 40 | 60 |
| 50 | 40 | 60 |
| 52 | 90 | 10 |
| 60 | 90 | 10 |

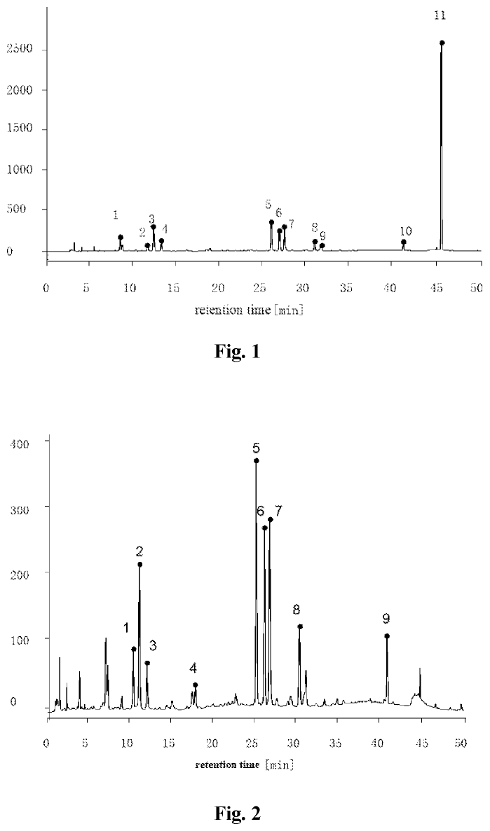

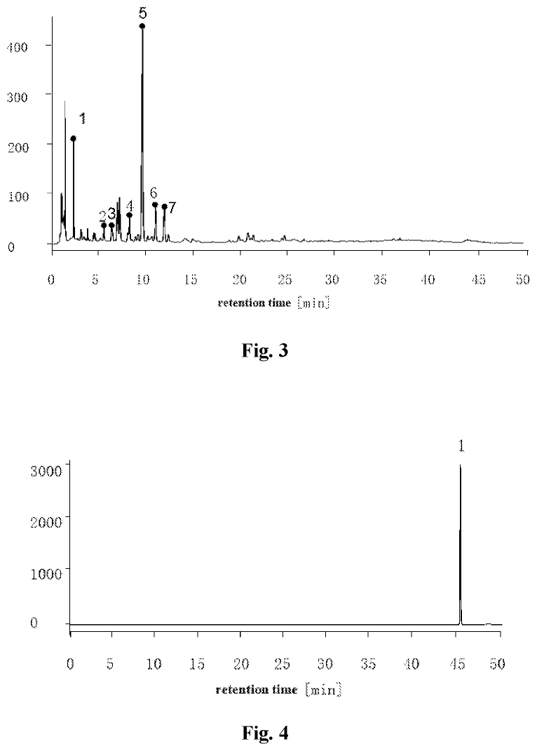

The fingerprint spectrum (see FIG. 1) of the traditional Chinese medicine formula composition for improving depression emotion and sleep obtained by the above high performance liquid chromatography method comprises 11 common fingerprint peaks, which are:

Peak 1 having an average retention time RT of 8.291 min, an RSD of 1.06%, and an average peak area of 1389.931, which accounts for 3.481% of the peak area;

Peak 2 having an average retention time RT of 11.481 min, an RSD of 1.43%, and an average peak area of 828.957, which accounts for 2.076% of the peak area;

Peak 3 having an average retention time RT of 12.173 min, an RSD of 0.92%, and an average peak area of 2552.112, which accounts for 6.391% of the peak area;

Peak 4 having an average retention time RT of 13.061 min, an RSD of 1.01%, and an average peak area of 1198.181, which accounts for 3.001% of the peak area;

Peak 5 having an average retention time RT of 25.763 min, an RSD of 1.23%, and an average peak area of 3074.151, which accounts for 7.698% of the peak area;

Peak 6 having an average retention time RT of 26.717 min, an RSD of 0.95%, and an average peak area of 2400.017, which accounts for 6.010% of the peak area;

Peak 7 having an average retention time RT of 27.303 min, an RSD of 0.86%, and an average peak area of 2736.155, which accounts for 6.852% of the peak area;

Peak 8 having an average retention time RT of 30.777 min, an RSD of 0.98%, and an average peak area of 1,260.393, which accounts for 3.156% of the peak area;

Peak 9 having an average retention time RT of 31.521 min, an RSD of 0.87%, and an average peak area of 811.315, which accounts for 2.032% of the peak area;

Peak 10 having an average retention time RT of 41.037 min, an RSD of 0.6%, and an average peak area of 735.617, which accounts for 1.842% of the peak area; and

Peak 11 having an average retention time RT of 45.426 min, an RSD of 0.5%, and an average peak area of 15062.947, which accounts for 37.721% of the peak area.

6. Identification of active ingredients of the traditional Chinese medicine formula composition for improving depression emotion and sleep

In the present invention, two target ingredients in the reference fingerprint spectrum of the traditional Chinese medicine formula composition were identified by measuring the retention time of an active ingredient reference substance. The active ingredient reference substance was determined by high performance liquid chromatography as follows:

1) The active ingredient reference substance: hyperoside (Y04A9X62302) and paeonol (1101708-201407) were both purchased from National Institutes for Food and Drug Control.

2) Preparation of the active ingredient reference solution: appropriate amounts of the two reference substances were taken respectively, and 50% methanol aqueous solution was added to the measuring flask, sonicated for 2 min, and diluted to the mark.

3) Liquid chromatographic conditions and determination methods were the same as those in “(1) Determination of a reference fingerprint spectrum of the traditional Chinese medicine formula composition for improving depression emotion and sleep”.

Through the comparison of spectrums, it can be known that among the 13 characteristic absorption peaks of the traditional Chinese medicine formula composition for improving depression emotion and sleep of the present invention, the peaks having retention times of 26.717 min and 45.426 min are the corresponding active ingredients, which are hyperoside and paeonol, respectively.

### Example 6 Determination of the Fingerprint Spectrum of Hypericum perforatum Extract

1. Reagents: acetonitrile (HPLC, Wokai); methanol (HPLC, Wokai); phosphoric acid was analytically pure; and UPLC purified water was self-made by Milli-Q Pure Water System.

2. Instrument: Agilent Infinity 1260 (CA, USA).

3. Preparation of a sample of Hypericum perforatum extract: 0.71 g of Hypericum perforatum extract was accurately weighed and placed into a 100 mL triangular conical flask, and 25 mL of 50% methanol was added, weighed, ultrasonically extracted for 20 min, and then re-weighed. 50% methanol was added to make up the lost weight, filtered, and then a certain volume of filtrate was taken and passed through a 0.45 m microfiltration membrane to obtain the sample solution of Hypericum perforatum extract.

4. Chromatographic conditions: the chromatographic conditions and determination methods are the same as those in “(1) Determination of a reference fingerprint spectrum of the traditional Chinese medicine formula composition for improving depression emotion and sleep” in Example 5.

5. Determination: The above sample solution of Hypericum perforatum extract was taken and injected into the liquid chromatograph, and the fingerprint spectrum of Hypericum perforatum extract was obtained according to the high performance liquid chromatography method. There are 9 absorption peaks of the main chemical ingredients, which are:

Peak 1 having an average retention time RT of 11.591 min, an RSD of 0.95%, and an average peak area of 1303.790, which accounts for 3.863% of the peak area.

Peak 2 having an average retention time RT of 12.299 min, an RSD of 0.86%, and an average peak area of 2686.452, which accounts for 9.402% of the peak area;

Peak 3 having an average retention time RT of 13.189 min, an RSD of 1.12%, and an average peak area of 895.530, which accounts for 3.134% of the peak area;

Peak 4 having an average retention time RT of 18.793 min, an RSD of 1.03%, and an average peak area of 538.669, which accounts for 1.885% of the peak area;

Peak 5 having an average retention time RT of 25.879 min, an RSD of 0.87%, and an average peak area of 4029.912, which accounts for 14.104% of the peak area;

Peak 6 having an average retention time RT of 26.838 min, an RSD of 0.92%, and an average peak area of 3122.853, which accounts for 10.930% of the peak area;

Peak 7 having an average retention time RT of 27.434 min, an RSD of 0.87%, and an average peak area of 3520.778, which accounts for 12.322% of the peak area;

Peak 8 having an average retention time RT of 30.911 min, an RSD of 1.01%, and an average peak area of 1604.837, which accounts for 5.617% of the peak area; and

Peak 9 having an average retention time RT of 41.111 min, an RSD of 0.78%, and an average peak area of 1040.855, which accounts for 3.643% of the peak area.

### Example 7 Determination of the Fingerprint Spectrum of Acanthopanax senticosus

1. The reagents and instruments in this Example were the same as those in Example 5.

2. Preparation of a sample of Acanthopanax senticosus extract: 0.23 g of Acanthopanax senticosus extract was accurately weighed and placed into a 100 mL triangular conical flask, and 25 mL of 50% methanol was added, weighed, ultrasonically extracted for 20 min, and then re-weighed. 50% methanol was added to make up the lost weight, filtered, and then a certain volume of filtrate was taken and passed through a 0.45 m microfiltration membrane to obtain the sample solution of Acanthopanax senticosus extract.

3. Chromatographic conditions: the chromatographic conditions and determination methods were the same as those in “(1) Determination of a reference fingerprint spectrum of the traditional Chinese medicine formula composition for improving depression emotion and sleep” in Example 5.

4. Determination: The above sample solution of Acanthopanax senticosus extract was taken and injected into the liquid chromatograph, and the fingerprint spectrum of Acanthopanax senticosus extract was obtained according to the high performance liquid chromatography method. There are 7 absorption peaks of the main chemical components, which are:

Peak 1 having an average retention time RT of 3.869 min, an RSD of 1.12%, and an average peak area of 583.333, which accounts for 4.466% of the peak area;

Peak 2 having an average retention time RT of 6.993 min, an RSD of 0.95%, and an average peak area of 272.368, which accounts for 2.085% of the peak area;

Peak 3 having an average retention time RT of 7.777 min, an RSD of 0.85%, and an average peak area of 296.854, which accounts for 2.273% of the peak area;

Peak 4 having an average retention time RT of 9.625 min, an RSD of 0.96%, and an average peak area of 361.169, which accounts for 2.765% of the peak area;

Peak 5 having an average retention time RT of 10.930 min, an RSD of 0.88%, and an average peak area of 3627.005, which accounts for 27.767% of the peak area;

Peak 6 having an average retention time RT of 12.300 min, an RSD of 0.85%, and an average peak area of 726.493, which accounts for 5.562% of the peak area; and

Peak 7 having an average retention time RT of 13.186 min, an RSD of 0.90%, and an average peak area of 750.645, which accounts for 5.747% of the peak area.

### Example 8 Methodological Verification of Quality Control of the Traditional Chinese Medicine Formula Composition for Improving Depression Emotion and Sleep

### 1. Preparation of Reference Solution

An appropriate amount of hyperoside was taken and dissolved in methanol to prepare a reference solution with a concentration of 1 mg/mL, 0.5 mg/mL, 0.25 mg/mL, 0.125 mg/mL, 0.0625 mg/mL, and 0.03125 mg/mL.

An appropriate amount of paeonol reference substance was taken and dissolved in methanol to prepare a reference solution with a concentration of 1 mg/mL, 0.5 mg/mL, 0.25 mg/mL, 0.125 mg/mL, 0.0625 mg/mL, and 0.03125 mg/mL.

### 2. Production of Standard Curve and Determination of Linear Range

An appropriate amount of hyperoside standard solution was taken and filtered through a 0.45 μm microfiltration membrane. 10 μL was injected, respectively, based on the set chromatographic conditions. The linear regression equation and linear range of hyperoside were obtained by drawing the standard curve with the chromatographic peak area as the ordinate and the concentration as the abscissa.

The regression equation of hyperoside is Y=10335.76985X-77.2047, R²=0.99985; and the regression equation of paeonol is: y=46735.024x-302.7421 (R²=0.99993). There is a good linear relationship between the concentration and the peak area of hyperoside and paeonol in the linear range.

### 3. Precision Experiment

The above hyperoside standard solution and paeonol standard solution were precisely taken and determined by HPLC, with consecutive injected 5 times for 10 μL each time, and determined for the peak area RSD value to test the precision of the high performance liquid chromatograph. For hyperoside, the RSD of the retention time is 0.06% and the RSD of the peak area is 1.07%; and for paeonol, the RSD of the retention time is 0.03% and the RSD of the peak area is 0.23%.

### 4. Stability Experiment

Samples of the traditional Chinese medicine composition were taken and determined by HPLC at 0 h, 3 h, 6 h, 9 h, 12 h, and 15 h respectively according to the above chromatographic conditions, with an injection volume of 10 μL. The peak area was integrated to calculate the relative standard deviation of the integrated value and test the stability. The RSD of the peak area of hyperoside was 0.35% and the RSD of the peak area of paeonol was 0.08%.

### 5. Reproducibility Experiment

The sample from the same batch was taken to prepare a tested solution according to the preparation method of the tested solution, and determined by HPLC according to the above chromatographic conditions, with an injection volume of 10 μL. The peak area was integrated to calculate the relative standard deviation and test the reproducibility. For hyperoside, the RSD of the retention time is 0.08% and the RSD of the peak area is 0.28%; and for paeonol, the RSD of the retention time is 0.06% and the RSD of the peak area is 0.11%.

### 6. Sample Recovery Experiment

5 samples of the traditional Chinese medicine composition with known content were weighed, and a certain amount of hyperoside and paeonol reference substances was added respectively. Then 5 samples were prepared according to the preparation method of the tested solution, and each sample was continuously injected with an injection volume of 10 μL for each injection. The content of hyperoside and paeonol was determined by the standard curve method, and the average recovery rate of the reference substance was calculated, respectively. The RSD of hyperoside content is 0.52%; and the RSD of paeonol content is 0.19%.

### Comparative Example 1

Compared with Example 5, other conditions remain unchanged, except for mobile phase and elution gradient:

Mobile phase: 0.1% formic acid aqueous solution (A)-acetonitrile (B); and

Elution gradient: 0-10 min, 5%-10% B; 10-20 min, 10%-20% B; 20-30 min, 20%-35% B; 30-40 min, 35%-55% B; 40-45 min, 55%-95% B; 45-50 min, 95% B; 50-55 min, 95%-5% B; and 55-65 min, 5% B.

However, the resolution of the chromatographic peak obtained under this condition was low, and the quality control of the traditional Chinese medicine composition could not be achieved.

### Comparative Example 2

Compared with Example 5, other conditions remain unchanged, except for the gradient conditions:

0.1% phosphoric acid aqueous solution (A)-acetonitrile (B); and

0-20 min, 10%-20% B; 20-30 min, 20%-25% B; 30-40 min, 25%-40% B; 40-50 min, 40%-10% B; and 50-60 min, 10% B.

However, the resolution of the chromatographic peak obtained under this condition was low, and the quality control of the traditional Chinese medicine composition could not be achieved.

### Comparative Example 3

Compared with Example 5, other conditions remain unchanged, except for the column temperature and elution conditions:

Column temperature: 25° C.;

Mobile phase: 0.1% phosphoric acid aqueous solution (A)-acetonitrile (B); and

Gradient: 0-20 min, 10%-20% B; 20-30 min, 20%-25% B; 30-40 min, 25%-40% B; 40-50 min, 40%-10% B; and 50-60 min, 10% B.

However, the resolution of the chromatographic peak obtained under this condition was low, and the quality control of the traditional Chinese medicine composition could not be achieved.

### Comparative Example 4

Compared with Example 5, other conditions remain unchanged, except for flow rate, mobile phase and elution gradient:

Flow rate: 0.8 ml/min,

Mobile phase: 0.1% formic acid aqueous solution (A)-acetonitrile (B); and

Elution gradient: 0-10 min, 5%-10% B; 10-20 min, 10%-20% B; 20-30 min, 20%-35% B; 30-40 min, 35%-55% B; 40-45 min, 55%-95% B; 45-50 min, 95% B; 50-55 min, 95%-5% B; and 55-65 min, 5% B.

However, the resolution of the chromatographic peak obtained under this condition was low, and the quality control of the traditional Chinese medicine composition could not be achieved.

### Comparative Example 5

Compared with Example 5, other conditions remain unchanged, except for mobile phase and elution gradient:

Mobile phase: water (A)-acetonitrile (B); and

Elution gradient: 0-10 min, 10%-14% B; 10-20 min, 14%-20% B; 20-30 min, 20%-25% B; 30-40 min, 25%-40% B; 40-42 min, 40%-60% B; 42-50 min, 60% B; 50-52 min, 60%-10% B; and 52-60 min, 10% B.

However, the resolution of the chromatographic peak obtained under this condition was low, and the quality control of the traditional Chinese medicine composition could not be achieved.

In addition, various other separation conditions were also tried in the present invention and the obtained chromatographic peak resolutions were low, and the quality control of the traditional Chinese medicine composition could not be achieved.

The foregoing description is merely preferred embodiments of the present invention and it should be noted that several improvements and modifications can be made by those ordinary skilled in the art without departing from the principles of the present invention, and these improvements and modifications should also be deemed to be within the protection scope of the present invention.

1. A method of establishing a fingerprint spectrum of a traditional Chinese medicine composition with effect of regulating depression emotion or a formulation thereof, wherein the traditional Chinese medicine composition consists of the following traditional Chinese medicine extracts in parts by weight: 40˜80 parts of Hypericum perforatum extract, 10˜40 parts of Acanthopanax senticosus extract, and 2˜20 parts of tree peony root bark extract; and
the fingerprint spectrum is constructed respectively by high performance liquid chromatography for a tested solution and an reference solution and the chromatographic conditions are as follows: an octadecylsilane bonded silica gel chromatographic column is used as a chromatographic column; column temperature is 25˜35° C.; flow rate is 0.5˜1.1 mL/min; detection wavelength is 290˜310 nm; mobile phase A is 0.05%˜0.15% phosphoric acid aqueous solution, mobile phase B is acetonitrile, and gradient elution program is as follows:

| time/min | A/% | B/% |
|---|---|---|
| 0 | 90 | 10 |
| 8 | 86 | 14 |
| 12 | 86 | 14 |
| 20 | 80 | 20 |
| 30 | 75 | 25 |
| 40 | 60 | 40 |
| 42 | 40 | 60 |
| 50 | 40 | 60 |
| 52 | 90 | 10 |
| 60 | 90 | 10 |

2. The method according to claim 1, wherein the detection wavelength is 300 nm.

3. The method according to claim 1, wherein the mobile phase A is 0.1% phosphoric acid aqueous solution.

4. The method according to claim 1, wherein the chromatographic column is Agilent TC-C18 chromatographic column of 250×4.6 mm, 5 m.

5. The method according to claim 1, wherein the column temperature is 30° C.

6. The method according to claim 1, wherein the flow rate is 1.0 mL/min.

7. The method according to claim 1, wherein the tested solution is prepared by a method comprising steps of: mixing the traditional Chinese medicine composition or the formulation thereof with 40 vt %˜60 vt % methanol aqueous solution with a ratio of the traditional Chinese medicine composition or the formulation thereof to the methanol aqueous solution in g/mL of (0.5˜2.0): 25, performing ultrasonic treatment and filtration, and collecting subsequent filtrate;
and the reference solution is prepared by a method comprising steps of: mixing hyperoside reference substance or paeonol reference substance with 40 vt %˜60 vt % methanol aqueous solution with a ratio of the hyperoside reference substance or the paeonol reference substance to the methanol aqueous solution in g/mL of (0.5˜1.5):1.

8. The method according to claim 1, wherein the traditional Chinese medicine composition consists of the following traditional Chinese medicine extracts in parts by weight: 65˜80 parts of Hypericum perforatum extract, 10˜35 parts of Acanthopanax senticosus extract, and 2˜10 parts of tree peony root bark extract.

9. The method according to claim 1, wherein the traditional Chinese medicine composition is prepared by a method comprising steps of:
(1) to Hypericum perforatum, adding 10˜12 times of weight of 60 vt %˜80 vt % ethanol aqueous solution containing 0.05%-2% sodium hydroxide, extracting under heating and refluxing 2 times for 1.5˜2.0 hours each time, combining the two filtrates and filtering, concentrating the filtrate to an extract with a relative density of 1.11˜1.13 at 72° C., and performing spray-drying to obtain Hypericum perforatum extract;
(2) to Acanthopanax senticosus pieces, adding 8˜10 times of weight of 0 vt %˜80 vt % ethanol aqueous solution, extracting under heating and refluxing 3 times for 1.0˜2.5 hours each, combining the three filtrates and filtering, concentrating the filtrate to an extract with a relative density of 1.11˜1.13 at 72° C., and performing spray-drying to obtain Acanthopanax senticosus extract;
(3) to tree peony root bark, adding 10˜14 times its weight of water, heating and recovering 8˜10 times its amount of distillate, refrigerating for 20˜24 hours, crystallizing, filtering, and low temperature drying to obtain tree peony root bark extract; and
(4) mixing the Hypericum perforatum extract, the Acanthopanax senticosus extract and the tree peony root bark extract according to a formula atio.

10. The method according to claim 1, wherein the traditional Chinese medicine composition is prepared by a method comprising steps of:
(1) to Hypericum perforatum, adding 10 times of weight of 80 vt % ethanol aqueous solution containing 0.10% sodium hydroxide, extracting under heating and refluxing 2 times for 1.5 hours each time, combining the two filtrates and filtering, concentrating the filtrate to an extract with a relative density of 1.12 at 72° C., and performing spray-drying to obtain Hypericum perforatum extract;
(2) to Acanthopanax senticosus pieces, adding 10 times of weight of the aqueous solution, extracting under heating and refluxing 3 times for 1.0 hours each time, combining the three filtrates and filtering, concentrating the filtrate to an extract with a relative density of 1.12 at 72° C., and performing spray-drying to obtain Acanthopanax senticosus extract;
(3) to tree peony root bark, adding 14 times of weight of water, heating and recovering 9 times of amount of distillate, refrigerating for 24 hours, crystallizing, filtering, and performing low temperature drying to obtain tree peony root bark extract; and
(4) mixing the Hypericum perforatum extract, the Acanthopanax senticosus extract and the tree peony root bark extract according to a formula ratio.
